(19) United States
(12) Patent Application Publication
(10) Pub. No.: US 2009/0060960 A1
(43) Pub. Date: Mar. 5, 2009

(54) Preparation containing barium sulfate

(76) Inventors: Petra Biehl, Neuss (DE); Reiner Mertscheit, Moers (DE); Heiner Schulte, Soest (DE); Shirley Shen, Shanghai (CN); Bernd Hirthe, Tonisvort (DE)

Correspondence Address: FULBRIGHT & JAWORSKI, LLP, 666 FIFTH AVE, NEW YORK, NY, 10103-3198, US

(21) Appl. No.: 11/988,591

(22) PCT Filed: Jul. 6, 2006
(86) PCT No.: PCT/EP2006/063945

§ 371 Date: Sep. 2, 2008

(30) Foreign Application Priority Data
Jul. 18, 2005 (DE) 102005034005.9

(51) Int. Cl. A61K 8/02; A61Q 17/04; A61Q 19/08; B32B 5/16

(52) U.S. Cl. 424/401; 424/709; 424/59; 428/402

(57) ABSTRACT

The invention relates to a preparation containing barium sulfate, a method for the production thereof, and the use thereof.

The present invention provides a preparation containing barium sulfate, a process for the production thereof and the use of this preparation.

The present invention provides in particular a preparation for topical application containing barium sulfate, preferably a cosmetic or dermatological formulation, a process for the production thereof and the use of this preparation, preferably as an anti-wrinkle cream.

Wrinkles in human skin are perceived optically because the possibility of diffuse light scattering is severely limited in them, which makes the actual wrinkles appear darker than the surrounding skin surface. For aesthetic reasons, attempts are often made to reduce or smooth out these wrinkles or at least to cover them optically. For this purpose, there are many different care products, some of which also contain barium sulfate (EP-A-1437117, EP-A-1369411, DE-A-10063433, WO-A-0192157).

Barium sulfate is on the one hand cheaply available and on the other hand it can be incorporated into care products without any problems.

However, the barium sulfate used in the aforementioned preparations has a particle size of less than 100 nm, preferably less than 50 nm (WO-A-0192157), or an average particle size of less than 100 nm, preferably between 5 and 50 nm, particularly between 15 and 30 nm (EP-A-1437117, EP-A-1369411, DE-A-10063433). However, barium sulfate with an average particle size of 100 nm only has a low relative scattering power for visible light, for example 0.01 for light with a wavelength of 550 nm. For smaller particle sizes, the scattering power for light at this wavelength is even lower. The use of barium sulfate having these particle sizes therefore requires the addition of other, mostly expensive, active substances and additives to the preparations so that these are suitable to reduce or smooth out wrinkles or to cover them optically.

The object of the present invention is to overcome the disadvantages of the prior art. In particular, it is the object of the present invention to provide a cosmetic preparation which contains barium sulfate and which is suitable to reduce or smooth out wrinkles or to cover them optically. It is a further object of the present invention to provide a cosmetic preparation which contains barium sulfate and is also suitable to reduce or smooth out wrinkles or to cover them optically without expensive active ingredients and additives. It is a further object of the present invention to provide a cosmetic preparation which contains barium sulfate and is suitable to reduce or smooth out wrinkles or to cover them optically over the entire spectrum of visible light.

According to the invention, this problem is surprisingly solved by the features of the main claim. Preferably embodiments are found in the subclaims.

As already known from WO-A-0192157, barium sulfate (both synthetic and natural) is capable of scattering, reflecting and refracting light as a result of its optical properties. Wrinkles appear darker than the surrounding skin surface owing to the limited diffuse light scattering. Particularly through the scattering power of barium sulfate, these wrinkles are perceived optically to a lesser extent.

The relative scattering power of barium sulfate particles is, however, heavily dependent on the average particle size. Thus, the relative scattering power for visible light at a wavelength of 550 nm is only 0.01 at 0.01 μm, already 0.04 at 0.2 μm and then at an average particle size of about 1 μm it reaches the maximum of 0.14, and then falls back to 0.06 at 10 nm and about 0.035 at 20 nm. Corresponding relationships can be seen for other ranges of visible light, with shorter-wave light being scattered more markedly on smaller particles and longer-wave light more readily on larger particles.

Surprisingly, it has been found that the object is achieved by the use of barium sulfate with a particle size of 1 to 30 μm. These barium sulfate particles can be both post-treated and untreated. Moreover, it has surprisingly been found that the problem is solved by the use of barium sulfate with a particle size of 0.1 to 8 μm. In this case, the barium sulfate particles are preferably post-treated.

The post-treatment can be both inorganic or organic and inorganic and organic in nature. The term ‘post-treatment’ is to be understood particularly as coating.

The inorganic coating preferably takes place with Al₂O₃, TiO₂, ZrO₂, Fe₂O₃, CeO₂, MnO₂, SiO₂ or mixtures of these and/or the corresponding hydroxides or mixtures of these. The proportion of the inorganic coating is 1 to 50 wt. %, preferably 5 to 30 wt. %, based in each case on the barium sulfate used.

The organic coating preferably takes place with stearic acid, silicone oils, simethicones and/or silanes. The proportion of the organic coating is 1 to 20 wt. %, preferably 2 to 10 wt. %, based in each case on the barium sulfate used.

In addition, both the untreated and the inorganically and/or organically post-treated [barium sulfate] can be mixed with inorganic substances, preferably inorganic oxides such as Al₂O₃, TiO₂, ZrO₂, Fe₂O₃, CeO₂, MnO₂, SiO₂ or mixtures of these and/or the corresponding hydroxides or mixtures of these. The proportion of these inorganic additives is 1 to 50 wt. %, preferably 5 to 30 wt. %, based in each case on the barium sulfate used.

The barium sulfate according to the invention is contained in application concentrations of 1 to 15 wt. %, preferably of 1 to 10 wt. %, particularly preferably of 1 to 6 wt. %, especially preferably of 1 to 4 wt. %, especially of 1 to 3 wt. % of the preparation. Other application concentrations that are advantageous according to the invention are 6 to 15 wt. %, preferably 6 to 12 wt. %, particularly preferably 7 to 10 wt. %.

The barium sulfate used according to the invention here can be of natural and/or synthetic origin.

The production of the preparation according to the invention takes place by processes which are known from the prior art.

In addition, it has surprisingly been found that barium sulfate can be used not only in anti-wrinkle creams but, according to the invention, in a wide variety of care products. The care products can be based on an emulsion; they can be based both on an oil-in-water and on a water-in-oil formulation. In particular, daily care products, eye creams, sun protection care products, body care products, anti-cellulite products, makeup formulations, foundations and powders can be mentioned here as examples; all these products can also contain—where appropriate—a UV protection agent.

The present invention provides in detail:
a preparation for topical application, which:
   contains barium sulfate;
   contains barium sulfate, wherein the barium sulfate can be of natural or synthetic origin;
   contains barium sulfate, wherein the barium sulfate has a particle size of 1 to 30 μm, preferably of 1 to 20 μm, particularly preferably of 1 to 10 μm, especially preferably of 1 to 5 μm;
   contains barium sulfate, wherein the barium sulfate has a particle size of 0.1 to 8 μm, preferably of 0.15 to 5 μm, particularly preferably of 0.2 to 2 μm;
   contains barium sulfate, wherein the barium sulfate is untreated;
   contains barium sulfate, wherein the barium sulfate is inorganically and/or organically post-treated;
   contains barium sulfate, wherein the barium sulfate is coated with Al₂O₃, TiO₂, ZrO₂, Fe₂O₃, CeO₂, MnO₂, SiO₂ or mixtures (of) two or more of these and/or the corresponding hydroxides or mixtures (of) two or more of these;
   contains barium sulfate, wherein the proportion of the inorganic coating is 1 to 50 wt. %, preferably 5 to 30 wt. %, based in each case on the barium sulfate used;
   contains barium sulfate, wherein the barium sulfate is coated with one or more organic substances from the group of stearic acid, silicone oils, simethicones and silanes or mixtures (of) two or more of these;
   contains barium sulfate, wherein the proportion of the organic coating is 1 to 20 wt. %, preferably 2 to 10 wt. %, based in each case on the barium sulfate used;
   contains barium sulfate, wherein the barium sulfate is mixed with inorganic substances, preferably inorganic oxides such as Al₂O₃, TiO₂, ZrO₂, Fe₂O₃, CeO₂, MnO₂, SiO₂ or mixtures (of) two or more of these and/or the corresponding hydroxides or mixtures (of) two or more of these;
   contains barium sulfate, wherein the proportion of the inorganic additives is 1 to 50 wt. %, preferably 5 to 30 wt. %, based in each case on the barium sulfate used;
   contains barium sulfate, wherein the barium sulfate is contained in the preparation in an application concentration of 1 to 15 wt. %, preferably of 1 to 10 wt. %, particularly preferably of 1 to 6 wt. %, especially preferably of 1 to 4 wt. %, especially of 1 to 3 wt. %;
   contains barium sulfate, wherein the barium-sulfate is contained in the preparation in an application concentration of 6 to 15 wt. %, preferably of 6 to 12 wt. %, particularly preferably of 7 to 10 wt. %;
   contains barium sulfate, wherein the preparation is based on an emulsion;
   contains barium sulfate, wherein the preparation is based on an oil-in-water formulation;
   contains barium sulfate, wherein the preparation is based on a water-in-oil formulation;
   contains barium sulfate, wherein the preparation is a cosmetic or dermatological formulation, preferably a daily care product, an anti-wrinkle preparation, an eye cream, a sun protection care product, a body care product, an anti-cellulite product, a makeup formulation, a foundation and/or a powder;
   contains barium sulfate, wherein the preparation contains a UV protection agent;
a process for the production of a preparation, wherein barium sulfate is added to the to the components, which are known per se, for conventional cosmetic preparations;
use of the preparations according to the invention as a cosmetic or dermatological formulation, preferably as a daily care product, anti-wrinkle preparation, eye cream, sun protection care product, body care product, anti-cellulite product, in a makeup formulation, foundation and/or powder.
use of barium sulfate in preparations for topical application, preferably cosmetic or dermatological formulations, particularly preferably in daily care products, anti-wrinkle preparations, in eye creams, in sun protection care products, in body care products, in anti-cellulite products, in makeup formulations, in foundations and/or in powders.

The invention is explained below by examples, without being limited thereto:

### EXAMPLE 1

### Preparation and Composition of a Barium Sulfate Coated with TiOOH and Al(OH)₃

200 g of BaSO₄ with an average particle size of 4 μm are stirred into about one litre of partially demineralised water using a straight-arm paddle agitator. The suspension is heated to 80° C. 192 ml of an aqueous, sulfuric acid titanyl sulfate solution (42.75 g TiOSO₄; 51.8 g H2SO₄) are added slowly (5 ml/min) with stirring. During the addition, the pH value is kept at a constant pH 7 with the aid of 25% sodium hydroxide solution.

66.7 ml of sodium aluminate solution (content corresponds to 300 g Al₂O₃ per litre) are then added slowly (5 ml/min) at a constant pH. During the addition, the pH value is kept at a constant pH 7 with the aid of sulfuric acid. On completion of the addition, stirring is performed for two hours at pH 7 and 80° C. The material is then filtered by suction and the filter cake is washed to a filtrate conductivity of less than 100 μS/cm and dried in a drying cabinet at 105° C.

The product can then be tempered for a further 30 minutes in a muffle furnace at 800° C.

Composition of the product (wt. %):

79.3% BaSO₄, 8.6% TiOOH, 12.1% Al(OH)₃

### EXAMPLE 2

### Preparation and Composition of a Barium Sulfate Coated with TiOOH, Al(OH)₃ and Fe(OH)₃

200 g of BaSO₄ with an average particle size of 4 μm are stirred into about one litre of partially demineralised water using a straight-arm paddle agitator. The suspension is heated to 80° C. 192 ml of an aqueous, sulfuric acid titanyl sulfate solution (42.75 g TiOSO₄; 51.8 g H₂SO₄) are added slowly (5 ml/min) with stirring. During the addition, the pH value is kept at a constant pH 7 with the aid of 25% sodium hydroxide solution. 75.9 ml of an aqueous FeCl₃ solution (content corresponds to 200 g FeCl₃ per litre) are then added slowly (5 ml/min), with stirring. During the addition, the pH value is again kept at a constant pH 7 with the aid of 25% sodium hydroxide solution.

After adding the FeCl₃ solution, 66.7 ml of sodium aluminate solution (content corresponds to 300 g Al₂O₃ per litre) are added slowly (5 ml/min) at a constant pH. During the addition, the pH value is kept at a constant pH 7 with the aid of sulfuric acid. On completion of the addition, stirring is performed for two hours at pH 7 and 80° C. The material is then filtered by suction and the filter cake is washed to a filtrate conductivity of less than 100 μS/cm and dried in a drying cabinet at 105° C.

The product can then be tempered for a further 30 minutes in a muffle furnace at 800° C.

Composition of the product (wt. %):

76.3% BaSO₄, 8.2% TiOOH, 11.7% Al(OH)₃, 3.8% Fe(OH)₃.

### EXAMPLE 3

### Preparation and Composition of a Barium Sulfate Coated with TiO₂ and Al(OH)₃

200 g of BaSO₄ with an average particle size of 4 μm are stirred into about one litre of partially demineralised water using a straight-arm paddle agitator. The suspension is heated to 80° C. 100 ml of an aqueous TiO₂ suspension (content corresponds to 200 g per litre with an average primary particle size of the TiO₂ of 35 nm) are added slowly (5 ml/min) with stirring. 66.7 ml of sodium aluminate solution (content corresponds to 300 g Al₂O₃ per litre) are then added slowly (5 ml/min) at a constant pH. During the addition, the pH value is kept at a constant pH 7 with the aid of sulfuric acid. On completion of the addition, stirring is performed for two hours at pH 7 and 80° C. The material is then filtered by suction and the filter cake is washed to a filtrate conductivity of less than 100 μS/cm and dried in a drying cabinet at 105° C.

Composition of the product (wt. %):

79.8% BaSO₄, 8.0% TiO₂, 12.2% Al(OH)₃.

### EXAMPLE 4

### Preparation and Composition of a Barium Sulfate Coated with TiO₂, Al(OH)₃ and Fe(OH)₃

200 g of BaSO₄ with an average particle size of 4 μm are stirred into about one litre of partially demineralised water using a straight-arm paddle agitator. The suspension is heated to 80° C. 100 ml of an aqueous TiO₂ suspension (content corresponds to 200 g per litre with an average primary particle size of the TiO₂ of 35 nm) are added slowly (5 ml/min) with stirring. 75.9 ml of an aqueous FeCl₃ solution (content corresponds to 200 g FeCl₃ per litre) are then added slowly (5 ml/min), with stirring. During the addition, the pH value is kept at a constant pH 7 with the aid of 25% sodium hydroxide solution.

After adding the FeCl₃ solution, 66.7 ml of sodium aluminate solution (content corresponds to 300 g Al₂O₃ per litre) are added slowly (5 ml/min) at a constant pH. During the addition, the pH value is kept at a constant pH 7 with the aid of sulfuric acid. On completion of the addition, stirring is performed for two hours at pH 7 and 80° C. The material is then filtered by suction and the filter cake is washed to a filtrate conductivity of less than 100 μS/cm and dried in a drying cabinet at 105° C.

Composition of the product (wt. %):

76.7% BaSO₄, 7.7% TiO₂, 11.7% Al(OH)₃, 3.8% Fe(OH)₃. EXAMPLE 5

### Preparation and Composition of a Barium Sulfate Coated with TiOOH

200 g of BaSO₄ with an average particle size of 4 μm are stirred into about one litre of partially demineralised water using a straight-arm paddle agitator. The suspension is heated to 80° C. 192 ml of an aqueous sulfuric acid titanyl sulfate solution (42.75 g TiOSO₄; 51.8 g H₂SO₄) are added slowly (5 ml/min) with stirring. During the addition, the pH value is kept at a constant pH 7 with the aid of 25% sodium hydroxide solution.

On completion of the addition, stirring is performed for two hours at pH 7 and 80° C. The material is then filtered by suction and the filter cake is washed to a filtrate conductivity of less than 100 μS/cm and dried in a drying cabinet at 105° C.

Composition of the product (wt. %):

90.2% BaSO₄, 9.8% TiOOH.

### EXAMPLE 6

### Preparation and Composition of a Barium Sulfate Coated with TiOOH and Fe(OH)₃

200 g of BaSO₄ with an average particle size of 4 μm are stirred into about one litre of partially demineralised water using a straight-arm paddle agitator. The suspension is heated to 80° C. 192 ml of an aqueous, sulfuric acid titanyl sulfate solution (42.75 g TiOSO₄; 51.8 g H₂SO₄) are added slowly (5 ml/min) with stirring. During the addition, the pH value is kept at a constant pH 7 with the aid of 25% sodium hydroxide solution. 37.9 ml of an aqueous FeCl₃ solution (content corresponds to 200 g FeCl₃ per litre) are then added slowly (5 ml/min), with stirring. During the addition, the pH value is kept at a constant pH 7 with the aid of 25% sodium hydroxide solution.

On completion of the addition, stirring is performed for two hours at pH 7 and 80° C. The material is then filtered by suction and the filter cake is washed to a filtrate conductivity of less than 100 μS/cm and dried in a drying cabinet at 105° C.

The product can then be tempered for a further 30 minutes in a muffle furnace at 700° C.

Composition of the product (wt. %):

88.3% BaSO₄, 9.5% TiOOH, 2.2% Fe(OH)₃.

### EXAMPLE 7

### Composition and Preparation of an Anti-Wrinkle Cream on an Oil-in-Water Basis (Data in wt. %)

| Component | Ingredient | Proportion | |
|---|---|---|---|
| I | Caprylyl methicone | | 1.00% |
| | Cetearyl alcohol | | 0.50% |
| | Cetyl phosphate | | 1.00% |
| | Glyceryl stearate | | 0.50% |
| | Isopropyl palmitate | | 7.00% |
| | Mineral oil | | 8.00% |
| II | Ammonium acryloyl dimethyl | | 1.20% |
| | taurate/VP copolymer | | |
| III | Barium sulfate | | 5.00% |
| IV | Glycerol | | 5.00% |
| | Sodium lauroyl glutamate | | 0.50% |
| | Water | make up to | 100.00% |
| V | Tocopheryl acetate | | 1.00% |

| | |
|---|---|
| Parfum | as required |
| Preservatives | as required |

Component I is melted at about 80° C. Component II is then stirred into I. Component III is then dispersed into the mixture of components I and II while still hot. Component IV is then added immediately and stirring is continued until the mixture has cooled. Finally, component V is stirred into the mixture at 30° C.

1-21. (canceled)

22. A preparation for a topical application containing barium sulfate, wherein the barium sulfate with a particle size of 1 to 30 μm

23. A preparation according to claim 22, wherein the particle size is from 1 to 20 μm.

24. A preparation according to claim 22, wherein the barium sulfate is of natural or synthetic origin.

25. A preparation according to claim 22, wherein the barium sulfate is inorganically or organically post-treated.

26. A preparation according to claim 22, wherein the barium sulfate is coated with at least one of Al₂O₃, TiO₂, ZrO₂, Fe₂O₃, CeO₂, MnO₂, SiO₂ or a corresponding hydroxide thereof.

27. A preparation according to claim 26, wherein the proportion of the coating is 1 to 50 wt. % based in each case on the barium sulfate used.

28. A preparation according to claim 22, wherein the barium sulfate is coated with at least one organic substance selected from the group consisting of stearic acid, silicone oil, a simethicone and a silane or mixtures thereof.

29. A preparation according to claim 28, wherein the proportion of the coating is 1 to 20 wt. % based on the barium sulfate.

30. A preparation according to claim 22, wherein the barium sulfate is mixed with an inorganic oxide selected from the group consisting of Al₂O₃, TiO₂, ZrO₂, Fe₂O₃, CeO₂, MnO₂, SiO₂ or mixtures thereof or the corresponding hydroxide thereof.

31. A preparation according to claim 22, wherein the proportion of the inorganic additives is 1 to 50 wt. % based on the barium sulfate.

32. A preparation according to claim 22, wherein the barium sulfate is untreated.

33. A preparation according to claim 22, wherein the barium sulfate is contained in the preparation in an application concentration of 1 to 15 wt. %.

34. A preparation according to claim 22, wherein the barium sulfate is contained in the preparation in an application concentration of 6 to 15 wt. %.

35. A preparation according to claim 22, wherein the preparation is an emulsion.

36. A preparation according to claim 22, wherein the preparation is an oil-in-water formulation.

37. A preparation according to claim 22, wherein the preparation is a water-in-oil formulation.

38. A preparation according to claim 22, wherein the preparation is a cosmetic or dermatological formulation.

39. A preparation according to claim 22, wherein the preparation further comprises a UV protection agent.

40. A process for the production of a preparation according to claim 22, wherein barium sulfate is added to a component that is conventional for a cosmetic preparation.

41. A cosmetic or dermatological formulation comprising the preparation of claim 22.

42. A cosmetic or dermatological formulation according to claim 41, wherein the cosmetic or dermatological preparation is an anti-wrinkle preparation, an eye cream, a sun protection product, a body care product, in anti-cellulite product, a makeup formulation, an foundation or a powder.

43. A preparation for a topical application containing barium sulfate, wherein the barium sulfate has a particle size of 0.1 to 20 μm.

44. A preparation according to claim 43, wherein the barium sulfate is of natural or synthetic origin.

45. A preparation according to claim 43, wherein the barium sulfate is inorganically or organically post-treated.

46. A preparation according to claim 43, wherein the barium sulfate is coated with at least one of Al₂O₃, TiO₂, ZrO₂, Fe₂O₃, CeO₂, MnO₂, SiO₂ or a corresponding hydroxide thereof.

47. A preparation according to claim 43, wherein an inorganic coating is provided and is present in an amount of 1 to 50 wt. % based on weight of the barium sulfate.

48. A preparation according to claim 43, wherein the barium sulfate is coated with at least one organic substance selected from the group consisting of stearic acid, a silicone oil, simethicone and a silane.

49. A preparation according to claim 48, wherein the proportion of the coating is 1 to 20 wt. % based on the weight of the barium sulfate.

50. A preparation according to claim 43, wherein the barium sulfate is mixed with an inorganic oxide selected from the group consisting of Al₂O₃, TiO₂, ZrO₂, Fe₂O₃, CeO₂, MnO₂, SiO₂ or a corresponding hydroxide thereof.

51. A preparation according to claim 43, wherein an inorganic additive is present in the preparation in an amount of 1 to 50 wt. % based on the weight of barium sulfate.

52. A preparation according to claim 43, wherein the barium sulfate is untreated.

53. A preparation according to claim 43, wherein the barium sulfate is contained in the preparation in an application concentration of 1 to 15 wt. %.

54. A preparation according to claim 43, wherein the barium sulfate is contained in the preparation in an application concentration of 6 to 15 wt. %.

55. A preparation according to claim 43, wherein the preparation is an emulsion.

56. A preparation according to claim 43, wherein the preparation is an oil-in-water formulation.

57. A preparation according to claim 43, wherein the preparation is a water-in-oil formulation.

58. A preparation according to claim 43, wherein the preparation is a cosmetic or dermatological formulation.

59. A preparation according to claim 43, wherein the preparation further comprises a UV protection agent.

60. A process for the production of a preparation according to claim 43, wherein barium sulfate is added to a conventional component for a cosmetic preparation.

61. A cosmetic or dermatological formulation comprising the preparation of claim 43.

62. The cosmetic or dermatological formulation of claim 43, wherein the cosmetic or dermatological preparation is an anti-wrinkle preparation, an eye cream, a sun protection product, a body care product, in anti-cellulite product, a makeup formulation, an foundation or a powder.

63. A preparation according to claim 22, wherein the particle size ranges from 1 to 10 μm.

64. A preparation according to claim 22, wherein the particle size ranges from 1 to 5 μm.

65. A preparation according to claim 43, wherein the particle size ranges from 0.15 to 15 μm

66. A preparation according to claim 65, wherein the particle size ranges from 0.2 to 2 μm.
